(19) United States
(12) Patent Application Publication
(10) Pub. No.: US 2018/0296461 A1
(43) Pub. Date: Oct. 18, 2018

(54) ARSENIC-CHELATING COMPOSITION

(71) Applicant: CYRA CONSULTORA LIMITADA, Santiago (CL)

(72) Inventors: Carolina Valeska URRUTIA ALVAREZ, Santiago (CL); Carolina Andrea MORAGA OYARCE, Santiago (CL)

(21) Appl. No.: 15/569,383

(22) PCT Filed: May 6, 2016
(86) PCT No.: PCT/CL2016/050021

§ 371 Date: Oct. 25, 2017

(30) Foreign Application Priority Data
May 8, 2015 (CL) 1254-2015

(51) Int. Cl. A61K 8/96; A61K 8/73; A61K 8/98; A61Q 19/10; A61Q 5/02; A61Q 5/12; A61Q 11/00; B01J 20/24; B01J 20/16; C11D 3/22; C11D 3/12; C11D 3/384

(52) U.S. Cl. CPC A61K 8/965; A61K 8/73; A61K 8/982; A61Q 19/10; A61Q 5/02; C11D 3/384; A61Q 11/00; B01J 20/24; B01J 20/165; C11D 3/222; C11D 3/128; A61Q 5/12

(57) ABSTRACT

The invention relates to a biodegradable chemical composition with a chelating effect on arsenic, lead and mercury, comprising: (i) between 20 and 30 weight % of chitosan (poly-D-glucosamine); (ii) between 40 and 60 weight % of natural zeolite; and (iii) between 20% and 30 weight % of eggshell membrane. In a preferred embodiment, the composition comprises: 25 weight % of chitosan (poly-D-glucosamine); (ii) 50 weight % of natural zeolite; and (iii) 25 weight % of eggshell membrane. The invention also relates to all products used for personal hygiene, cleanliness and care that can include the biodegradable chemical composition as an additional component of the product.

### TECHNICAL FIELD

The present invention relates to a biodegradable chemical composition with a chelating effect on arsenic, lead and mercury. Said composition can be part of hygiene, cleaning and personal care products, without losing its effectiveness. Its use is mainly recommended for people living in areas with significantly high concentrations of arsenic, lead and mercury in the environment, who may be in dermal contact with environmental contaminants or through the intake of contaminated food.

### DESCRIPTION OF THE STATE OF THE TECHNIQUE

The strong human development that has occurred in the last hundred years in the world is indisputable. Such development encompasses the broadest fields, such as social development, industrial development and technological development. However, many industrial and social advances, leave a mark that is reflected in the same humanity. It is widely recognized that industrial development has left a contaminating trail that for a long time was not considered as a relevant element affecting human life. The same technological progress has generated a social development that at the time was not considered. It is worth mentioning the great population growth that important cities have experienced, due originally to the need for labor to support this industrial advance. Also, important population settlements have developed around industrial sectors, without taking into consideration some environmental elements that have manifested themselves over the population, for example, around the mining industry, mining camps were originally established, which over the years have become cities, which carry the environmental problems of the location and those that have been added as an effect of industry itself. Undoubtedly, all human development has generated harmful pollutants that affect, human life in different ways for example: air pollution, water pollution, noise pollution, pollution by solid waste and others. Thus we have different types of contamination, generated by the development of humanity, which have caused an alteration or contamination of natural resources, in particular, water for human consumption has been the natural resource whose quality has been most strongly affected.

Today, the problem of environmental pollution, because of its direct impact on human life, is a subject of great importance and concern for various countries and health organizations.

Although there is a wide range of pollutants that can enter the environment, the substances that have generated large pockets of pollution and poisoning in surrounding communities are mainly arsenic (As), lead (Pb) and mercury (Hg), Which according to the World Health Organization (WHO) correspond to toxic elements abundant in the earth's crust.

Arsenic is a metalloid that enters the environment naturally, through the atmosphere, groundwater and surface water in an amount of 8,000 tons per year, and also through human activity in an approximate amount of 23,600 tons per Year, surpassing, in this way, the natural contributions by almost three times. The main sources of arsenic contamination are brought about through mining, the use of fossil fuels, pesticides, herbicides, desiccants and wood preservatives (O'neill P. (1990). Arsenic. In: Heavy metals in soils. New York, USA 83-99).

According to the WHO, the countries most affected by arsenic exposure are Argentina, Bangladesh, Chile, China, India, Mexico and the United States of America, where it has been estimated that at least four million people have been exposed to levels above 50 mg/L of arsenic, which exceeds the standard of 0.01 mg/L proposed by WHO and adopted by the EPA (Environmental Protection Agency) from 1997 (Bundschuh J.; Garcia, M. E. and Birkle, P. (2006). “Rural Latin America: a forgotten part of the global groundwater arsenic problem”).

Results with laboratory animals indicate that As^{III} is more toxic than As^{V} because pentavalent compounds have less effect on enzymatic activities (The National Academy of Sciences, 1999, Arsenic in drinking water. National Academy Press, Washington).

The toxicity of arsenic is mainly due to the inhibition of sulfhydryl-containing enzymes found in keratin, which is why, after 2 to 4 weeks of continuous exposure to arsenic, they can be found in the hair, nails and skin, arsenic being the only metal that shows a clear affinity for this type of tissue, especially the skin. In turn, it has been shown that arsenic can accumulate and penetrate human skin and that dermal absorption through human skin depends largely on its speciation, thus, as As^{III} is more liposoluble than As^{V}, it can enter with greater speed, therefore, it can be absorbed in a more rapid form by the skin (Ouypornkochagorn, Feldmann. 2010. Dermal uptake of arsenic through human skin depends strongly on its speciation. Environ Sci Technol. May 15; 44(10):3972-8. doi: 10.1021/es903667y).

The most common ways of exposure to arsenic are ingestion and inhalation. Dermal exposure is more relevant in populations settled on territories with geological data of arsenic, such as Antofagasta, Chile.

Lead is a heavy metal that is widely distributed in the environment. Most comes from activities such as mining, industrial manufacturing and burning fossil fuels. Lead has many different uses. It is used in the manufacture of batteries, ammunition, metal products (welding and plumbing) and X-ray protective films. Due to concerns about public health, the amount of lead in paints and ceramics and materials for caulking and soldering has declined considerably in recent years. The use of lead as an additive in gasoline was banned in 1996 in the United States. (Corey, G. Plomo. Mexico, ECO/OPS. 1989, Serie de Vigilancia 8. 103 p.).

The precipitation of atmospheric lead in the water represents a source of contamination of flora and fauna. The natural contents of lead in water vary from 0.5 to 1 μg/L. In water contaminated by man, lead levels can vary widely, sometimes exceeding the limit value established by WHO (50 μg/L). The usual forms of lead in water are carbonates and sulfates, which tend to be insoluble in water but are bioaccumulated by aquatic organisms. The lead deposited in the soil comes mainly from the air and is responsible for the lead content in the dust from the streets and from the homes, which is mainly in contact with people's skin. (Corey, G. Plomo. Mexico, ECO/OPS. 1989, Serie de Vigilancia 8. 103 p.).

Lead can be inhaled and absorbed through the respiratory system or ingested and absorbed through the gastrointestinal tract; Percutaneous absorption of inorganic lead is minimal, but organic lead is well absorbed through this route. After absorption, lead is distributed in compartments, first circulating in blood attached to the red blood cells, 95% of the lead is attached to the erythrocyte, then distributed to the soft tissues such as liver, kidney, bone marrow and central nervous system that are the target organs of toxicity, after 1 to 2 months the lead diffuses to the bones where it bioaccumulates (Melinda M. Valdivia Infantas. Intoxicación por plomo. Rev. Soc. Per. Med. Inter. 18(1) 2005).

The toxic mechanism of lead is given in three modes: one is competing with essential metals, especially calcium and zinc at their insertion sites; Another is by their affinity for the sulfhydryl (—SH) groups of proteins, which means alteration of the form and function of them, and given that numerous proteins, including several enzymes, have —SH groups, physiological alterations can be very generalized in the organism; Finally, it also acts by altering the transport of essential ions. Its inhibiting power of numerous enzymes forms the basis of the various toxic effects that are reflected in the clinic and laboratory. From this, the most widely recognized and studied biochemical and clinical expression is the alteration of the synthesis of the heme group, in the hematopoietic process. Numerous tissues and parenchyma are affected by lead. One of the most affected systems of interest is the central nervous system, where lead poisoning in children generates psychological and behavioral alterations. The damage to the neurons is direct and is accompanied by a significant deficit in their metabolic energy. Segmental demyelination and axonal degeneration occur in the peripheral nervous system; The resulting diffuse polyneuropathy may evolve into paralysis of some nerves. There is evidence that lead may have carcinogenic, genotoxic and teratogenic effects on mankind (Corey, G. Plomo. Mexico, ECO/OPS. 1989, Serie de Vigilancia 8. 103 p.).

Mercury is an element that is naturally present in the Earth's crust, it can come from volcanic activity, rock erosion or human activity. The latter is the main cause of mercury emissions, mainly from the combustion of coal in power plants, heating and cooking, industrial processes, waste incineration and mining of mercury, gold and other metals. (Gaioli, et al., Impacto del mercurio sobre la salud humana y el ambiente, Arch Argent Pediatr 2012; 110(3):259-264/259).

Mercury enters through respiratory, digestive and cutaneous routes. The respiratory route is by inhalation. In occupational health this route is the most important and, both elemental and inorganic mercury and their compounds, can enter by inhalation and reach the blood with an efficiency of 80%. The digestive route is by ingestion. In the gastrointestinal tract, inorganic mercury is absorbed in an amount less than 10.01%, probably due to its inability to react with biologically important molecules, by forming macromolecules which hinder its absorption and because it undergoes an oxidation process. Inorganic mercury compounds (salts) are absorbed at between 2 and 15%, depending on their solubility. While, in contrast, the absorption of the organic compounds by this route is 95%, regardless of whether the methyl radical is bound to a protein or not. The cutaneous route is by contact. Cases of intoxication by topical application of compounds containing methylmercury have been reported. However, this pathway has not been shown to play an important role in occupational exposure, when compared with the others (Augusto V Ramírez. Intoxicación ocupacional por mercurio. An Fac med. 2008; 69(1):46-51).

After inhalation or ingestion of various mercury compounds or after skin exposure to them, neurological and behavioral disorders can be observed, with symptoms such as tremors, insomnia, memory loss, neuromuscular effects, headaches or cognitive and motor dysfunction. In workers exposed for several years at atmospheric levels of at least 20 μg/m3 of elemental mercury, mild subclinical signs of central nervous system toxicity can be observed. Effects on the kidneys ranging from proteinuria to renal failure have been reported. (OMS. El mercurio y la salud. Centro de prensa. Septiembre de 2013. Nota descriptiva No 361).

With respect to the previously described arsenic, lead and mercury, there is currently no effective method to reduce toxicities due to the high mobility and chemical changes of these heavy metals in the environmental cycle.

Since there is no curative treatment to control diseases caused by arsenic, lead and mercury contamination, the emphasis should be placed on prevention, particularly in preventing the ingestion, contact or absorption of arsenic present in the environment by individuals potentially exposed to it.

According to what is described in the Chilean patent application No. 965-2012, numerous techniques have been developed for the removal of arsenic from water. Arsenic removal technologies can be divided into four main groups, they are:

1.—Oxidation/precipitation: in which the oxidation can be with air or chemical. This methodology is characterized by being simple, low cost, allows the removal of arsenic in situ and oxidizes other organic and inorganic compounds. In turn, chemical oxidation allows the elimination of microbes. Although this methodology has the disadvantage of removing only As^{V} and requires a complementary process of precipitation.
2.—Coagulation/Coprecipitation: this technique includes coagulation with aluminum, coagulation with iron, and softening with lime. This methodology offers the advantage of the high availability of chemical reagents. But a major disadvantage of this method is the production of potentially toxic sludge, low efficiency in the removal of arsenic and generally requires previous stages such as sedimentation and filtration.
3.—Membrane Techniques: covers techniques of nanofiltration, reverse osmosis and electrodialysis. This technology allows a high removal capacity, does not produce toxic solid wastes, and other contaminants other than arsenic can be removed. The disadvantage of membrane-related techniques is the large loss of water, high operating costs and high maintenance technology.
4.—Sorption techniques: These techniques are highly efficient in removing pollutants and can be made with activated alumina, iron oxides or ion exchange resins.

Technical gaps for the environmental decontamination of sources contaminated with heavy metals, such as lead and mercury, in addition to arsenic, are known in the state of the art, but, no prior art document describes a natural and biodegradable composition, highly effective in the chelation of arsenic, lead and mercury. Thus Chinese document CN 101503255 describes a technology for treating the contamination of water bodies contaminated by mercury, lead, cadmium, copper, chromium, arsenic and other heavy metals by the use of a multi-compound sedimentation flocculating agent. Said multi-composite decantation flocculating agent is formed by the preparation of iron salt, aluminum salt, potassium permanganate, polyacrylamide and other solutions.

On the other hand, there is the European document EP 1046390, which describes an aqueous composition suitable for the removal of metals, metal ions, metal salts, environmental dirt and sebum from the hair, which comprises: an anionic dispersant; an effective amount of a reducing agent; an effective amount of a chelating agent; an effective amount of a scale inhibitor; an effective amount of a gelling agent; an effective amount of a surfactant component selected from the group consisting of anionic and amphoteric surfactants; and the rest is water.

Some methods and compositions intended to decontaminate arsenic-containing waters are also known in the state of the art, but none of them acts as a sequestering agent that may be in direct contact with the human body. Thus, Chilean patent application No. 965-2012 discloses a sorbent medium for the selective removal of contaminants from water, particularly arsenic, said sorbent medium consisting of a residual acrylic support material coated with iron oxides of Ferromagnetic characteristics, wherein the metal oxides are generated through an oxidation heat treatment and the acrylic support comprises waste or scraps of acrylic sheets for different uses such as advertising, construction, commercial display cabinets or decoration. The product of the mixture generates a thick paste, which is allowed to dry, then crush and sift the useful fraction comprising a particle size between 0.425 and 1.0 mm. It is then washed with deionized water to remove the excess iron salts.

On the other hand, Chinese publication CN 102614841 describes a method for the removal of arsenic from a body of water by the use of potassium permanganate (KMnO4) in modified egg shells. The method comprises preparing modified egg shells, whereby 1.5 to 2.5 g of pretreated eggshells are added in 100 ml of 0.01 to 0.05 mol/l of KMnO4 solution, stirring for 10 to 20 hours, and drying at a temperature of between 75 and 125° C. to obtain the modified egg shells. The pre-treatment of egg shells consists of washing the eggshells, removing the membranes adhered to the inner walls of egg shells, drying at a temperature between 75 and 125° C., grinding and sieving and with a 60 mesh screen. To remove the arsenic add 0.50 to 1.0 g of modified egg shells in 100 ml of 50 ppb of arsenic which is contained in waste water, stirring at normal temperature and at a frequency of 135 to 145 rpm for 5 to 24 hours.

It is also to be found that U.S. Pat. No. 7,378,372 describes a sorbent for the removal of at least one contaminant, which may be arsenic, from a fluid. Said sorbent is comprised of a first modified activated carbon, which contains a plurality of hydrated metal oxide particles substantially dispersed therein, and whose ability to remove arsenic is significantly higher as compared to a second sorbent comprising an activated carbon modified by a direct precipitation method which contains a plurality of hydrated metal oxide particles.

There is also in the state of the art information describing methods or products comprising zeolite or chitosan as an additional component for the treatment of water contaminated with arsenic and other heavy metals.

Thus, for example, Chinese patent CN 100419055 discloses a chitosan soap, which comprises between 0.05% and 12%, by weight, of chitosan or a derivative thereof, both soluble in water. This chitosan soap, is a product of low skin irritation, good cleaning power in general, can effectively maintain the moisture of the skin and delay the aging of the skin. People who wash or shower with chitosan soap can form complexes of heavy metals on the surface of the body, avoiding dermal contamination.

Also, U.S. Pat. No. 6,638,918 discloses a composition comprising chitosan in the form of a nanofiber web having an interstitial space of at least 80%. Wherein the chitosan has a specific surface area of at least 100 m²/g, a degree of crystallinity of less than 1% and is soluble in aqueous solutions having a pH between 1 and 6.3. Said composition can be used in hair care; for personal hygiene providing moisturizing benefits; for malodour control; in the inhibition of microbial contamination; for the care of the mouth or the denture including the improvement of the general state of health of gums and teeth; as a flocculant, coagulant or chelator in the purification of water and beverages; as a metal scavenger; and as a vehicle for the supply of medicines.

In relation to zeolite, U.S. Pat. No. 5,409,627, describes a bleaching particulate detergent composition, which comprises: (a) 15 to 50% by weight of a crystalline organic surfactant system; (B) 10 to 80% by weight of an alumino silicate comprising a zeolite P having a ratio of silicon to aluminum not higher than 1.33 (zeolite MAP); (C) a bleaching system comprising a bleaching compound of peroxide in an amount of 5 to 35% by weight, and a bleaching catalyst in an amount of 0.02 to 0.08% by weight, comprising a source of Mn and/or Fe ions and a ligand which is an organic macrocyclic compound.

Thus, in the prior art there is neither a biodegradable chemical composition having a chelating effect of lead, mercury and arsenic in water, or in another contaminated source or body, such as described in the present invention, which confers a preventive solution to dermal exposure, which can be used in cleaning products linked to daily activities, associated with hygiene and/or personal care, which will possess properties to reduce the toxic effect of lead, Mercury and arsenic from cooking water, shower, sink and dishwasher.

The composition described in the present invention is a safe, natural and biodegradable substance, highly effective in the chelation of arsenic, lead and mercury. This substance can be associated without difficulties or loss of effectiveness to hygiene, cleaning and personal care products such as bar soap, liquid soap, shampoo, toothpaste, creams, dishwashing liquid, laundry detergent, fruit and vegetable detergent and other products.

### SUMMARY OF THE INVENTION

The present invention relates to a biodegradable chemical composition with chelating effect of arsenic, lead and mercury, which comprises: (i) 20% to 30%, by mass, of chitosan Poly-D-glucosamine; (ii) 40% to 60%, by mass, of natural zeolite; And (iii) 20% to 30%, by mass, of eggshell membrane. Preferably, the composition comprises: (i) 25%, by mass, of chitosan Poly-D-glucosamine; (ii) 50% by mass of natural zeolite; and (iii) 25%, by mass, of eggshell membrane. The invention also includes all those products intended for hygiene, cleaning and personal care which may include the biodegradable chemical composition as an additional component of the product.

### DETAILED DESCRIPTION OF THE INVENTION

For a better understanding of the present invention, it should be understood that “% by mass” refers to the measurement of the amount of matter a body possesses, thus not using the concept of “weight”, which is a measure of the force that is caused on the body by the gravitational field.

For the present invention, when mentioning “eggshell”, it should be understood that all eggs from all types of birds are included, although due to their large population, domestication and food and industrial use, chicken eggs will be more common.

The present invention relates to a biodegradable chemical composition with the chelating effect on arsenic, lead and mercury that is present in the environment (water, air and soil) and may have contact with the skin of persons or foods consumed by persons. Said composition is comprised of: (i) 20% to 30%, by mass, of chitosan Poly-D-glucosamine; (ii) 40% to 60%, by mass, of natural zeolite; and (iii) 20% to 30%, by mass, of eggshell membrane.

Research already known has shown that some of the components which form part of the composition described in the present invention in the treatment of water contaminated with arsenic, lead and mercury have already been used independently of one another. But to date, no composition has been developed to include the three components mentioned above, which, through a synergistic effect, enhance its action as a chelating agent of arsenic, lead and mercury.

Preferably, the biodegradable chemical composition is comprised of: (i) 25%, by mass, of chitosan Poly-D-glucosamine; (ii) 50% by mass of natural zeolite; and (iii) 25%, by mass, of eggshell membrane.

It should be noted that the first component of the composition, chitosan Poly-D-glucosamine, is a natural polysaccharide which is obtained from the catalytic N-deacetylation of chitin, resulting in a macromolecule containing D-glucosamine units and N Acetyl-D-glucosamine. Poly-D-glucosamine chitosan with a degree of deacetylation between 70% and 95%, preferably a degree of deacetylation of 95%, is preferred in the particular case of the present invention. Deacetylation is an alkaline treatment at high temperatures where it is subjected to chitin to obtain chitosan. About 50% deacetylation and under 95% deacetylation, chitosan is obtained. The degree of deacetylation identifies the relative ratio of active amino groups in the chitosan polymer, thus, the greater the degree of deacetylation, the greater the retention of metals or metalloids.

Zeolites are a family of highly crystalline hydrated aluminosilicate minerals. In itself, zeolite is a mineral rock composed of aluminum, silicon and oxygen. It is found in a wide variety of regions of the world where prehistoric volcanic activity occurred near water, or where water has been present for millennia since eruptions. Zeolite was discovered in 1756 by the Swedish mineralogist Baron Axel Fredrick Cronstedt. Its structure forms cavities occupied by large ions and water molecules with great freedom of movement, which allows the ions adsorbed in the internal channels of the same, can be interchanged. Zeolite has a natural porosity because it has a crystalline structure with windows, cages, and superjails. Natural zeolites have windows of limited size (“pore size”) and all are hydrophilic (have an affinity for water). There are about 40 natural zeolites known in the world, of which only seven can be used in any industrial application, they are: clinoptilolite, which is the one of greater industrial application; Mordenite; Chabazite; Erionite; Ferrierite; Phillipsite; and analcima. Some synthetic zeolites with hydrophobic characteristics that have affinity for organic compounds and with little or no affinity for water have also been developed. Zeolite can adsorb molecules in its large internal area, as long as they can pass through the windows or pores. Zeolite can be used as a molecular sieve because it has a uniform window size (or pore). On the other hand, zeolite is metastable, which means that it is stable as long as it is maintained at a suitable temperature and pH. Within this range, it is not affected by large oscillations in temperature, pressure, or ionizing radiation. Natural zeolite exists as a natural rock, and can reach the size of a large rock. However, the crystals of synthesized zeolite always measure less than one millimeter, this is because these crystals grow very slowly. In this aspect, natural zeolites can and should be considered as materials with physical and chemical properties that can be modified, favoring the design and development of new materials and technologies.

In the present invention, to form the biodegradable chemical composition with a chelating effect on arsenic, lead and mercury, the zeolite to be employed is clinoptilolite-mordenite. Clinoptilolite-mordenite is the most abundant natural zeolite in nature and has a high cation exchange capacity and a high affinity for NH₄⁺ ions. Clinoptilolite-mordenite, like other zeolites, has a cage-like structure, consisting of SiO₄ and AlO₄ tetrahedra bound by shared oxygen atoms. The negative charges of the AlO₄ units are balanced with the presence of interchangeable cations, notably calcium, magnesium, sodium, potassium and iron. These ions can be displaced by other substances, for example heavy metals and ammonia ions. This phenomenon is known as cation exchange, and it is this ability of clinoptilolite-mordenite which gives it advantageous properties.

Eggshell constitutes the protective cover of the egg, the wall that defends it from the action of external agents, and the medium through which gaseous and liquid exchanges can be made with the surrounding environment. The shell represents between 9% and 12% of the weight of the egg; and is composed mainly of mineral substances, among which calcium carbonate (94%) is one of the most important as a structural component. There are other mineral principles in the shell, but in very small quantities, such as tricalcium phosphate and magnesium carbonate. The eggshell is porous (it can contain between 7,000 and 17,000 pores) and is not waterproof, therefore, it acts as a true coating.

In the shell of any type of egg, there are 2 interstitial membranes or films, which serve as a protective layer against microorganisms, to cover the albumen and separate it from the shell, and at the same time help to fix the various elements that make up the egg. There are two films: the external and the internal. The outer film covers the shell internally, while the inner film surrounds the albumen. Both remain in contact, except at the level of the air chamber located at the wide end of the egg. Such interstitial membranes or films are composed of collagen-type protein fibers, the films act as a high-efficiency filter-membrane type mechanical barrier. In addition, these films have an antibacterial action as a consequence of the lysozyme content. Both films offer a variable permeability, in order to allow breathing and the prolongation of life in the egg-cell. The two films are of a fibrous nature, and are in intimate contact when the external life of the egg begins, once it is laid; but later, in the zone corresponding to the major pole of the product, they are separated, and the space between the two membranes constitutes what is called the air chamber, whose volume increases with the days from the moment of the laying of the egg, and as a consequence of the gas exchange that takes place between him and the environment (Revista Cubana Aliment Nutr 2009; 19(1 Sup1):S84-S102. Dr. Sc. Jesús Valdés Figueroa).

Eggshells are generally considered to be a waste or solid residue from a variety of technical fields, such as egg incubation plants, industries that process eggs for other purposes (pastries, mayonnaise and sauces), to mention a few, and which, simply constitute a waste which one must get rid of.

In the present case, the investigators and inventors of the invention, by means of the research and development carried out with the various components of the eggshell, have been able to take advantage of a component that in another type of industry can be waste, As a primary agent and with an added value, to assist in the chelation of a contaminant present in water and to confer a benefit to human health.

For the case of the present invention, the eggshell is subjected to a pretreatment with 10% hydrochloric acid (HCl). Said pre-treatment is initiated by washing with abundant distilled water an amount of between 300 and 500 grams of eggshells, then the washed egg shells are immersed in a medium with 10% HCl at 37%. After 14 to 18 hours, once the majority of the calcium carbonate in the shell has been degraded, the acid is removed from the container and the membranes removed from the rest of the eggshell where it has not completely degraded the calcium carbonate. Subsequently, the membranes are washed with abundant distilled water. The membranes are then allowed to dry in an air oven set at a temperature of 80° C. for 4 to 5 hours. Finally, the egg shell membrane is milled to a particle size of 0.2 mm.

The biodegradable chelating chemical composition of arsenic, lead and mercury which is present in water, skin and food, as already mentioned, is preferably comprised of: (i) 25% by mass of chitosan Poly-D-glucosamine with 95% deacetylation Degree; (ii) 50%, by mass, of clinoptilolite-mordenite zeolite; And (iii) 25%, by mass, of pre-treated egg shell membrane with 10% HCl (37%). Therefore, to obtain 100 g of said composition, they measure the mass, on an analytical balance, separately of 25 g of poly-D-glucosamine chitosan with 95% deacetylation degree, 50 g of clinoptilolite-mordenite type zeolite and 25 g of eggshell membrane of pretreated with the procedure described in the previous paragraph. Finally, all the substances are mixed until a homogeneous mixture is reached.

Said biodegradable chemical composition with chelating effect on arsenic, lead and mercury, is preferably available in a solid state, particularly in the granular state. However, it can also be diluted in a suitable liquid medium and prepared in a liquid state.

The biodegradable chemical composition with the chelating effect on arsenic, lead and mercury, as described in the present invention, has a potent synergistic effect among the three components in the reduction of arsenic, lead and mercury. Presence of arsenic, lead and mercury wherein said water was treated with each of the components independently or with a combination of two of them, as shown in Table No. 1, it was found that the composition of the invention, which comprises the three components, shows percentages of reduction much higher than each component alone.

TABLE 1
Percentage of reduction of arsenic (As), lead (Pb) and mercury
(Hg) of the individual components, alone or in mixtures,
used to generate the composition of the invention and percentage
reduction of the composition of the invention.

| | Reduction | Reduction | Reduction |
|---|---|---|---|
| | of As in | of Pb in | of Hg in |
| Compound | water (%) | water (%) | water (%) |
| Chitosan | 34.39% | 96.34% | 98.27% |
| Zeolite | 0% | 98.76% | 60.70% |
| Eggshell membrane | 43.35% | 88.28% | 96.36% |
| 25% Chitosan + 75% Zeolite | 62.13% | 98.35% | 98.68% |
| Composition of the invention | 78.9% | 99.99% | 99.80% |

The advantages of the biodegradable chemical composition of the invention are as follows:
Innocuous for the health of living beings. It does not negatively affect the survival and development of the Danio rerio fish species, according to tests and data obtained in the bioassay laboratory, of the Milenio building of the University of Chile, and of the microalgae Chlamydomonas reinhardtii, according to tests and data obtained in the laboratory Of Cyra Consultora Ltda. In tables No 2 and No 3, results of toxicity bioassays are given for lettuce and microalgae seeds, where the safety of the composition of the present invention is demonstrated.
Sequesters over 75% of arsenic in water, significantly decreasing the toxicity of this metalloid. Data obtained from the company Hydrolab that complies with NCh. 17.025 and summarized in Table 1.
Sequesters over 99% lead in water, significantly reducing the toxicity of this heavy metal. Data obtained from the company Hydrolab that complies with NCh. 17.025 and summarized in Table 1.
Sequesters over 98% mercury in water, significantly decreasing the toxicity of this metalloid. Data obtained from the company Hydrolab that complies with NCh. 17.025 and summarized in Table 1.
Derived from natural organic and inorganic compounds and is environmentally friendly, as it is biodegradable and the eggshell is obtained from companies that dispose of it, such as bakeries.
Possible to combine it with hygiene, cleaning and personal care products without losing its effectiveness, which is a highly relevant and innovative hygiene and commercial advantage.

TABLE 2
Acute toxicity bioassay with Lettuce seeds in a medium of distilled
water (control group) or with the respective treatments.

| | | Hypocotyl | Reduction of |
|---|---|---|---|
| Samples | Germination (%) | reduction (%) | radicle (%) |
| Control Group | 60% | 0% | 0% |
| As (0.05 mg/L) | 50% | 18.76% | 29.66% |
| Composition | 60% | 0% | 0% |
| of the invention | | | |

TABLE 3
Bioassay of acute toxicity with microalgae in the TAP medium
(control group) or with the respective treatments.

| Samples | Chemical stress overgrowth (%) |
|---|---|
| Control Group | 0 |
| As (0.05 mg/L) | 83% |
| Composition of the invention | 0 |

In relation to the last of the advantages mentioned above (combination with hygiene, cleaning and personal care products without losing its effectiveness), effectively the biodegradable chemical composition with chelating effect on arsenic, lead and mercury, can be incorporated into hygiene, cleaning and personal care products, without losing or reducing its chelating properties on arsenic, lead and mercury. Thus, the composition of the invention may be a part of personal hygiene, cleaning and/or personal care products, such as bar soap, liquid soap, shower gel, shampoo, hair conditioner, toothpaste, mouthwash, disposable wet wipes for cleaning hard surfaces, disposable wet wipes for cleaning the body, dishwashing liquid, laundry detergent, fruit and vegetable disinfectant detergent, skin care cream, sun block cream, ie the composition of the invention may be part of all products of daily or common use in which humans can have direct contact with environments with arsenic, lead or mercury. So it is that the soaps (in bar and liquid) and the shampoo will be occupied in the daily water baths of the people; toothpaste in oral hygiene and its corresponding rinses, creams (for skin care and sun block) in daily and constant use and its contact with water each time hand and/or face grooming is performed; wet disposable wipes for cleaning hard surfaces or for the human body when necessary and in dishwashing liquid by the direct contact that will be had with the water occupied at the time of washing. The above listing of products can not be considered as limiting for the presence of the composition of the invention, it may be present in all known hygiene, cleaning and/or personal care products or in those which may be prepared in the future.

From all of the foregoing, then, all hygiene, cleaning and/or personal care products comprising the components of each product (surfactants, water, oils, solubilizers, preservatives, emollients, thickeners, glycerin, vaseline, carrier, colorants, flavorings, exfoliants, UV filters, specific chemical compounds, etc.) and the biodegradable chemical composition with chelating effect on arsenic described in the present invention.

Thus, there is a personal hygiene or cleansing product, which comprises: (i) from 70% and 99.5% by mass of a composition comprising a surfactant and other hygienic agents; and (ii) between 0.5% and 30%, by mass, of the biodegradable chemical composition with the chelating effect of arsenic, lead and mercury described in the present invention.

Where the product of personal hygiene or cleaning, can be, bar soap, liquid soap, shampoo, hair conditioner, exfoliating shower gel, vegetable disinfectant, toothpaste, mouthwash, dishwashing detergent or laundry detergent. Said products comprise a composition comprising a surfactant and other complementary hygienic agents, the latter of which may be: water, oils, solubilizers, preservatives, emollients, thickeners, colorants, flavorings, exfoliants, UV filters, antiseptics, pearlescent agent, pH regulators and specific chemical compounds. As already noted, the personal cleansing product further comprises the biodegradable chemical composition having the chelating effect on arsenic in water previously described in the present invention.

There is also a personal care product, comprising: (i) between 70% and 99.5%, by mass, of a composition comprising a base emulsion and other complementary ingredients; and (ii) between 0.5% and 30%, by mass, of the biodegradable chemical composition with a chelating effect on arsenic, lead and mercury described in the present invention.

Where the product for personal care, may be, skin care cream or sun block cream. Said products comprise a composition comprising a base emulsion formed by water and oil or water and silicone, and other complementary ingredients, among the latter may be: emulsifiers, preservatives, emollients, thickeners, colorants, flavorings, exfoliants, UV filters, antiseptics, pH regulators and other specific chemical compounds. As already noted, the personal care product further comprises the biodegradable chemical composition with the chelating effect on arsenic, lead and mercury, previously described in the present invention.

The scope of the biodegradable chemical composition with a chelating effect on arsenic, lead and mercury, described in the present invention, should not be limited to the products mentioned in the text itself, but should encompass all arsenic, lead and mercury chelating products, or a process for chelating or sequestering arsenic, lead and mercury, wherein the composition described above is included or used.

We claim:

1. A biodegradable chemical composition with a chelating effect on arsenic, lead and mercury, CHARACTERIZED in that it comprises: (i) 20% to 30% by weight of chitosan poly-D-glucosamine; (ii) 40% to 60% by weight of natural zeolite; and (iii) 20% to 30%, by weight of eggshell membrane.

2. The biodegradable chemical composition according to claim 1, CHARACTERIZED in that it comprises: (i) 25% by weight of chitosan poly-D-glucosamine; (ii) 50% by weight of natural zeolite; and (iii) 25% by weight of eggshell membrane.

3. The biodegradable chemical composition according to claim 1 or 2, CHARACTERIZED in that the chitosan poly-D-glucosamine has a deacetylation degree between 70% and 95%.

4. The biodegradable chemical composition according to claim 1 or 2, CHARACTERIZED in that the natural zeolite is clinoptilolite-mordenite.

5. The biodegradable chemical composition according to claim 1 or 2, CHARACTERIZED in that the eggshell has been pretreated with HCl.

6. The biodegradable chemical composition according to claim 5, CHARACTERIZED in that the eggshell has been pretreated with 10% HCl (37%).

7. The biodegradable chemical composition according to claim 1 or 2, CHARACTERIZED in that it is in granular form.

8. The biodegradable chemical composition according to claim 1 or 2, CHARACTERIZED in that it is in liquid form.

9. A personal care product, CHARACTERIZED in that it comprises: (i) between 70% and 99.5% by weight of a composition comprising a surfactant and other complementary hygienic agents; and (ii) between 0.5% and 30% by weight of the biodegradable chemical composition with chelating effect on arsenic, lead and mercury described in claims 1 to 8.

10. The personal care product according to claim 9, CHARACTERIZED in that the complementary hygienic agents are selected from: water, oils, solubilizers, preservatives, emollients, thickeners, colorants, flavorings, exfoliants, UV filters, antiseptics, pearlescent agent, pH regulators and specific chemical compounds.

11. The personal care product according to claim 9, CHARACTERIZED in that it is a bar soap, liquid soap, shampoo, hair conditioner, exfoliating shower gel, mouthwash, toothpaste, vegetable disinfectant, disposable wet wipes for cleaning hard surfaces, disposable wet wipes for body cleaning, dishwashing liquid or laundry detergent.

12. The personal care product according to claim 11, CHARACTERIZED in that it is a bar soap.

13. The personal care product according to claim 11, CHARACTERIZED in that it is a liquid soap.

14. The personal care product according to claim 11, CHARACTERIZED in that it is a shampoo.

15. The personal care product according to claim 11, CHARACTERIZED in that it is a hair conditioner.

16. The personal care product according to claim 11, CHARACTERIZED in that it is a exfoliating shower gel.

17. The personal care product according to claim 11, CHARACTERIZED in that it is a mouthwash.

18. The personal care product according to claim 11, CHARACTERIZED in that it is a toothpaste.

19. The personal care product according to claim 11, CHARACTERIZED in that it is a vegetable disinfectant.

20. The personal care product according to claim 11, CHARACTERIZED in that it corresponds to disposable wet wipes for cleaning hard surfaces.

21. The personal care product according to claim 11, CHARACTERIZED in that it corresponds to disposable wet wipes for body cleaning.

22. The personal care product according to claim 11, CHARACTERIZED in that it is a dishwashing liquid.

23. The personal care product according to claim 11, CHARACTERIZED in that it is a laundry detergent.

24. A personal care product, CHARACTERIZED in that it comprises: (i) between 70% and 99.5% by weight of a composition comprising a base emulsion and other complementary agents; and (ii) between 0.5% and 30% by weight of the biodegradable chemical composition with chelating effect on arsenic, lead and mercury described in claims 1 to 8.

25. The personal care product according to claim 24, CHARACTERIZED in that the complementary agents are selected from: emulsifiers, preservatives, emollients, thickeners, colorants, flavors, exfoliants, UV filters, antiseptics, pH regulators and other specific chemical compounds.

26. The personal care product according to claim 24, CHARACTERIZED in that it is a skin care cream or sun block cream.

27. The personal care product according to claim 26, CHARACTERIZED in that it is a skin care cream.

28. The personal care product according to claim 26, CHARACTERIZED in that it is a sun block cream.
